(19) United States
(12) Patent Application Publication
(10) Pub. No.: US 2016/0223526 A1
(43) Pub. Date: Aug. 4, 2016

(54) METHOD OF SCREENING FOR COMPOUNDS USEFUL IN THE TREATMENT OF HUNTINGTON DISEASE

(71) Applicant: INNOVATIVE CONCEPTS IN DRUG DEVELOPMENT, Gemenos (FR)

(72) Inventors: NATHALIE COMPAGNONE, AURIOL (FR); CLOTILDE BISCARRAT, LA CADIERE D'AZUR (FR)

(21) Appl. No.: 15/021,975

(22) PCT Filed: Sep. 22, 2014
(86) PCT No.: PCT/EP2014/070115

§ 371 Date: Mar. 15, 2016

(30) Foreign Application Priority Data
Sep. 20, 2013 (EP) 13306290.1

(51) Int. Cl. G01N 33/50

(52) U.S. Cl. CPC G01N 33/5079; G01N 33/5091; G01N 33/502; G01N 2800/2835; G01N 2800/52

(57) ABSTRACT

The present invention relates to a method of screening for compounds useful in the treatment of Huntington disease using a signature based on mitochondrial behaviour variables. The invention also relates to a method for diagnosing Huntington disease and a method for monitoring the response of a subject affected with Huntington disease to therapy.

### FIELD OF THE INVENTION

The present invention relates to the field of medicine, in particular to the diagnosis and treatment of Huntington disease.

### BACKGROUND OF THE INVENTION

Huntington disease (HD) is a progressive neurological disorder that leads to a distinctive chorea, cognitive loss, various psychological disorders, and eventually death. HD is caused by the expansion of an unstable polymorphic trinucleotide (CAG)n repeat in exon 1 of the huntingtin (Htt) gene, which translates into an extended polyglutamine tract in the protein. This mutation is autosomal and dominant, thus only one copy of the mutated or changed gene is sufficient to result in expression of the disease.

The Htt gene is a ubiquitous gene modulating several key functions in cells including transport, calcium homeostasis, neurotransmitter release, gene transcription, proteasome and mitochondrial function. From these complex molecular interactions derives a wide range of clinical patterns in the pathological progression of the disease.

When a patient suffers clinical signs that may resemble HD, a neurologist will diagnose the disease with a thorough neurologic examination using the HD unified rating scale further confirmed by a genetic testing demonstrating the presence of more than 39 CAG repeats in the Htt gene. However, because the penetration of the disease depends both on the length of the CAG repeat and on pre-existing conditions in which the mutated gene impacts cellular function in sensitive regions of the brain, this makes difficult to exclude expression of the disease in mutations with 35-39 repeats. Conversely one cannot be sure that a patient with a confirmed HD diagnosis with 39 repeats will develop HD. Furthermore, chorea is not specific to HD and may occur in several autoimmune disorders, genetic, or drug-related conditions. As a consequence, the differential diagnosis of HD-like syndromes is complex and may lead to unnecessary and costly investigations (Martino et al., 2012).

As HD diagnosis relies mostly on genetic testing, few studies exist to identify protein or imaging markers capable of identifying early progression signs of the disease in HD pre-symptomatic patients. As example, compounds capable of binding to TSPO (formerly known as the peripheral benzodiazepine receptor) have been postulated to be imaging markers for neurodegenerative diseases associated to inflammation including HD (e.g. the international patent application WO 10/020000; Politis et al., 2011). However, there is still a clear lack of markers that can be used to diagnose HD at early stages of disease progression or even in the asymptomatic stage, when treatment is more likely to prove beneficial to patients.

Furthermore, there is currently no approved drug for treating HD or slowing its progression. Benzodiazepines, neuroleptics, and antiepileptic medications may be used to control choreic symptoms. However, in addition to motor symptoms, HD patients show depression, bradykinesia, cognitive impairment, aggressive behavior and other complications such as eating disorders.

Because of the existence of the validated genetic testing, the prodromal stages of the disease are thought to be the best moment to act therapeutically. However, there is a need for validated markers indicative of biological activity of drugs at the prodromal stage. Similarly, a quantifiable and reliable biomarker for monitoring disease progression is crucial for clinical studies of neuroprotection, and this remains an area of active research. Understanding of the underlying pathophysiological mechanisms continues to grow, based mainly on cellular and animal models of HD.

Over the years, different models for HD have been described from insects (Drosophilae melanogaster) (Jackson et al., 1998), invertebrate models such as flatworms (C. elegans) (Faber et al., 2002; Parker et al., 2005), various rodent models (Carter et al., 1999; Tabrizi et al., 2000; Menalled et al., 2002; Wheeler et al., 2000; Hodgson et al., 1999; Gray et al., 2008), a transgenic ovine (Jacobsen et al., 2010), and pig model (Yang et al., 2010) to a recently developed non-human primate transgenic model (Yang et al., 2008).

These models have greatly contributed to the understanding of the pathophysiology of the disease but none of them fully recapitulate the human pathology. Furthermore, species differences are important and translation of drug efficacy results has thus been limited.

Therefore, there is a great need of in vitro human-derived models capable of recapitulating the fully complexity of the huntingtin proteomic interactome and that could be used to efficiently select drugs useful in the treatment of HD.

### SUMMARY OF THE INVENTION

Using molecular imaging of mitochondria in human live cells, the inventors identified a set of markers that is specific to HD and can be used to diagnose HD at early or asymptomatic stage of the disease. Furthermore, they demonstrated that the signature obtained with these markers can be reversed by treatments and thus provides a human-derived model to screen drugs capable of reversing disease progression of HD.

In a first aspect, the present invention relates to a method, preferably an in vitro method, of screening for compounds useful in the treatment of Huntington disease, wherein the method comprises

a) contacting living cells obtained from a sample from a subject affected with Huntington disease, with a test compound; and

b) measuring in said contacted cells, the values of the mitochondrial behaviour variables (i) to (vii):

(i) a variable selected from the group consisting of the average frequency of stops during trajectories of individual mitochondria (V1) and the average frequency of burst during displacement of individual mitochondria (V34), and a combination thereof;

(ii) the average number of individual mitochondria per unit of cell area, or a dispersion descriptor of the numbers of individual mitochondria per unit of cell area (V12);

(iii) the average area of mitochondria, or a dispersion descriptor of the areas of mitochondria (V13);

(iv) the frequency of mitochondria displaying an area between 0.51 and 1 μm² (V15);

(v) the frequency of mitochondria displaying an area between 10 and 20 μm² (V23);

(vi) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(vii) a variable selected from the group consisting of the average moving speed of mitochondria, or a dispersion descriptor of the moving speeds of mitochondria (V32) and the average maximal moving speed of individual mitochondria, or a dispersion descriptor of the maximal moving speeds of mitochondria (V33), and a combination thereof.

The method may further comprise comparing the values obtained in step b) with the values obtained in absence of the test compound.

The method may further comprise calculating a score for each variable using the following equation: score=(NC−Var)*100/(NC−PC), wherein NC is the value or average value obtained with HD sample(s) in absence of the test compound, PC is the value or average value obtained with healthy sample(s), and Var is the measured value of the variable. A test compound may be identified as useful in the treatment of Huntington disease when all measured variables have a positive score.

In a second aspect, the present invention also relates to a method, preferably an in vitro method, for diagnosing Huntington disease in a subject, wherein the method comprises:

a) measuring in living cells obtained from a sample from said subject the values of the mitochondrial behaviour variables (i) to (vii):

(i) a variable selected from the group consisting of the average frequency of stops during trajectories of individual mitochondria (V1) and the average frequency of burst during displacement of individual mitochondria (V34), and a combination thereof;

(ii) the average number of individual mitochondria per unit of cell area, or a dispersion descriptor of the numbers of individual mitochondria per unit of cell area (V12);

(iii) the average area of mitochondria, or a dispersion descriptor of the areas of mitochondria (V13);
(iv) the frequency of mitochondria displaying an area between 0.51 and 1 μm² (V15);

(v) the frequency of mitochondria displaying an area between 10 and 20 μm² (V23);

(vi) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(vii) a variable selected from the group consisting of the average moving speed of mitochondria, or a dispersion descriptor of the moving speeds of mitochondria (V32) and the average maximal moving speed of individual mitochondria, or a dispersion descriptor of the maximal moving speeds of mitochondria (V33), and a combination thereof.

The method may further comprise determining if said subject is affected with HD based on the measured values of mitochondrial behaviour variables.

Preferably, the subject comprises 36 to 39 CAG repeats in the htt gene.

The method may further comprise calculating the z-scores of measured variables. Preferably, positive z-scores of the 1^{st}, preferably V1, 2^{nd} and 4^{th} variables and negative z-scores of the 3^{rd}, 5^{th}, 6^{th}, preferably V29, and 7^{th}, preferably V32, variables are indicative that the subject suffers from Huntington disease.

In a third aspect, the present invention further relates to a method, preferably an in vitro method, for monitoring the response of a subject affected with Huntington disease to therapy, wherein the method comprises

a) measuring in living cells obtained from a sample from said subject, before and after the administration of the treatment, the values of the mitochondrial behaviour variables (i) to (vii):

(i) a variable selected from the group consisting of the average frequency of stops during trajectories of individual mitochondria (V1) and the average frequency of burst during displacement of individual mitochondria (V34), and a combination thereof;

(ii) the average number of individual mitochondria per unit of cell area, or a dispersion descriptor of the numbers of individual mitochondria per unit of cell area (V12);

(iii) the average area of mitochondria, or a dispersion descriptor of the areas of mitochondria (V13);

(iv) the frequency of mitochondria displaying an area between 0.51 and 1 μm² (V15);

(v) the frequency of mitochondria displaying an area between 10 and 20 μm² (V23);

(vi) a variable selected from the group consisting of the frequency of mitochondria displaying an area between 70 and 100 μm² (V29), the total area of regions containing entwined mitochondria to the total cell area (V7), and the frequency of mitochondria displaying an area between 100 and 200 μm² (V30), and any combination thereof; and

(vii) a variable selected from the group consisting of the average moving speed of mitochondria, or a dispersion descriptor of the moving speeds of mitochondria (V32) and the average maximal moving speed of individual mitochondria, or a dispersion descriptor of the maximal moving speeds of mitochondria (V33), and a combination thereof; and

b) comparing the measured values obtained in step a) for samples obtained before and after the administration of the treatment.

The method may further comprise calculating a score for each variable using the following equation: score=(NC−Var)*100/(NC−PC), wherein NC is the value obtained before the administration of the treatment, PC is the value or average value obtained with healthy sample(s), and Var is the measured value of the variable. Preferably, the patient is responsive to the therapy or is susceptible to benefit from the therapy when all measured variables have a positive score.

In particular, these methods may comprise measuring the mitochondrial behaviour variables V1, V12, V13, V15, V29 and V32, preferably V1, V12, V13, V15, V23, V29 and V32. They may also further comprises measuring at least one additional mitochondrial behaviour variable selected from the group consisting of V7, V30 V33 and V34.

These methods may further comprise comparing the measured values of mitochondrial behaviour variables with the values of said variables measured in a sample obtained from a healthy subject.

Preferably the sample is selected from the group consisting of skin biopsy sample, nervous tissue biopsy sample and serum or blood sample. More preferably, the sample is skin biopsy sample.

The cells may be selected from the group consisting of fibroblasts, induced pluripotent stem cells derived from fibroblasts, lymphocytes and neuronal cells. Preferably, the cells are fibroblasts.

Preferably, the dispersion descriptor is selected from the group consisting of the variance, the standard deviation and an interquantile range, preferably the interquartile range.

Preferably, before measuring the values of the mitochondrial behaviour variables, mitochondria contained in said living cells are labelled. Mitochondria may be labelled using any suitable label, preferably using a fluorescent label, and more preferably using MitoTracker Green.

The values of mitochondrial behaviour variables may be obtained from images captured using a fluorescence microscope or a differential interference-contrast (DIC) microscope coupled to a suitable image acquisition device, preferably a CCD camera. Preferably, the values of variables are obtained from images captured using a fluorescence microscope coupled to a CCD camera.

### BRIEF DESCRIPTION OF THE DRAWINGS

FIG. 1: Dendogram of the hierarchical cluster analysis applying the Ward's method.

FIG. 2: Heat map representation of the classification of the different subjects in the two subgroups identified by hierarchical clustering. The graph shows the z-scores values for the seven variables constituting the HD-signature.

FIG. 3: HD-signature of the invention shown by the z-score of each variable in healthy and diseased subject groups.

FIG. 4: Dose-response analysis of Resveratrol of the 7 different variables (V1, V32, V12, V13, V15, V23 and V29) of the HD-signature of the invention (% of phenotypic rescue).

FIG. 5: Weighted representation of the dose-response effect of Resveratrol on disease-modification.

FIG. 6: Dose-response analysis of Cyclosporine A on the 7 different variables (V1, V32, V12, V13, V15, V23 and V29) of the HD-signature of the invention (% of phenotypic rescue).

FIG. 7: Weighted representation of the dose-response effect of Cyclosporine A on disease-modification.

FIG. 8: Disease progression and schematic representation of the complexity of the different functional interactions resulting form the mutation of the single gene Htt.

### DETAILED DESCRIPTION OF THE INVENTION

Currently, both the normal function of Htt in neurons and the molecular mechanism by which the expanded polyQ sequence in Htt causes selective neurodegeneration remain elusive. However, it is known that the htt gene is a ubiquitous gene modulating several key functions in cells including transport, calcium homeostatis, neurotransmitter release, gene transcription, proteasome and mitochondrial function. From this complexity derives a wide range of clinical patterns in the pathological progression of the disease paralleling a variety of molecular events taking place in different cells that are affected with different susceptibility to these molecular modifications (FIG. 8). Current mechanistic approach to identify drugs with disease-modifying capabilities have focused on a single of these mechanisms hoping that it will be early enough in the chain of events to allow for modulation of downstream events. To date, this approach has not been successful.

The inventors previously described that analyzing the behaviour of an organelle, in particular mitochondrion, in a live cell can yield significant information to predict the effect of a compound on an animal or human organism. They defined said behaviour by analyzing the membrane permeability, the dynamic motility of the organelle inside the live cell through space and time, the dynamic changes occurring in the organelle morphology, and the interaction of the organelle with its cellular environment such as the dynamic relationship existing between organelle and elements of the cytoskeleton. This general strategy was disclosed in the U.S. Pat. No. 8,497,089, the content of which is herein enclosed in its entirety by reference. The inventors thus herein consider the global functionalities of the organelle and not solely the individual functions in cellular metabolism.

Because mitochondria are sensitive to the cellular environment and constantly communicate with this environment, they generate thousands of protein-protein interactions within the mitochondria and with other cellular organelles and compartments. Using molecular imaging of mitochondria in live cells, the inventors experimentally and dynamically assessed the mitochondrial reticular system in live cells allowing the capture of the resultant of those interactions that describe the mitochondrial behaviour. They measured 37 mitochondrial behaviour variables relating to the motility, the morphology, the network organization and the permeability of the mitochondria. Motility variables comprise, for example, measures of speed of displacement, amplitude, frequency and regularity of movements as well as distance traveled. Morphology variables comprise, for example, measures of mitochondrial dynamics (fusion-fission balance) and the frequency of apparition of various typical morphological features. The mitochondrial reticular network organization is scored with respect to its orientation, distribution and regionalization with respect to the intracellular cytoskeleton and/or to particular hot-spots in the cells such as the microtubule organizing center and focal adhesion points. Mitochondrial membrane permeability is measured by the dynamic analysis of signal intensity within individual mitochondria.

Based on this analysis, they identified a HD-signature comprising only seven mitochondrial behaviour variables that are sufficient to segregate healthy and HD patients. They further demonstrated that this signature can be pharmacologically reversed and can thus be used to screen compounds useful in the treatment of HD.

### DEFINITIONS

The term “Huntington disease”, “HD” or “Huntington chorea” (OMIM: #143100) refers to an autosomal dominant progressive neurodegenerative disorder caused by an expanded trinucleotide repeat (CAG)n, encoding glutamine, in the gene encoding huntingtin (Gene ID: 3064) on chromosome 4p16.3. In normal individuals, the number of repeats is from 6 to 35. Individuals with repeat number between 36 and 39 may develop HD and individuals with repeat number of 40 and above will develop HD.

As used herein, the term “subject” or “patient” refers to an animal, preferably to a mammal, even more preferably to a human, including adult, child and human at the prenatal stage.

As used herein, the term “HD patient” or “HD subject” refers to a subject having at least 36 CAG repeats in the Htt gene and who have developed or will develop HD. The subject may be at asymptomatic or symptomatic stage of the disease.

As used herein, the term “healthy patient” or “healthy subject” refers to a subject who is not affected with HD, in particular a subject having from 6 to 35 CAG repeats in the Htt gene. Preferably, the subject is not affected with any known disease, i.e. apparently healthy.

As used herein, the term “treatment”, “treat” or “treating” refers to any act intended to ameliorate the health status of patients such as therapy, prevention and retardation of HD. In certain embodiments, such term refers to the amelioration or eradication of HD or symptoms associated with HD, in particular neurological symptoms. In other embodiments, this term refers to minimizing the spread or worsening of HD resulting from the administration of one or more therapeutic agents to a subject affected with HD.

As used herein, the term “sample” means any sample containing living cells derived from a subject. Examples of such samples include fluids such as blood, plasma, saliva, urine and seminal fluid samples as well as biopsies, organs, tissues or cell samples. Preferably, the sample is selected from the group consisting of skin biopsy, nervous tissue biopsy, serum and blood. More preferably, the sample is a skin biopsy. The sample may be treated prior to its use. In particular, skin biopsies may be treated to isolate fibroblasts cells. Fibroblasts may be isolated using any method known by the skilled person. For example, the dermal component of the skin may be cut into small pieces and treated over night with collagenase type 1 and dispase. After centrifugation and resuspension, cells may be seeded in culture flasks and cultured in fibroblasts proliferation medium containing, for example, Dulbecco's minimum essential medium, 10% fetal calf serum, penicillin and streptomycin.

As used herein, the term “dispersion descriptor” refers to a measure of dispersion denoted how stretched or squeezed is a distribution of values. Preferably, this descriptor is selected from the group consisting of the variance, the standard deviation, and an interquantile range. Preferably, the interquantile range is the interquartile range or the interdecile range. The interquartile range is the difference between the upper and lower quartiles, i.e. between the 25^{th} percentile (splits lowest 25% of data) and the 75^{th} percentile (splits highest 25% of data). The interdecile range is the difference between the 1^{st} and the 9^{th} deciles, i.e. between the 10^{th} percentile (splits lowest 10% of data) and the 90^{th} percentile (splits highest 90% of data). In a preferred embodiment, the dispersion descriptor is selected from the group consisting of the variance, the standard deviation and the interquartile range. Methods for calculating these values are commonly known by the skilled person.

The methods of the invention as disclosed below, may be in vivo, ex vivo or in vitro methods, preferably in vitro methods.

In a first aspect, the present invention concerns a method of screening for compounds useful in the treatment of HD, wherein the method comprises

a) contacting living cells obtained from a sample from a subject affected with Huntington disease with a test compound; and

b) measuring in said contacted cells, the values of the mitochondrial behaviour variables (i) to (vii):

(i) a variable selected from the group consisting of V1: the average frequency of stops during trajectories of individual mitochondria, and V34: the average frequency of burst during displacement of individual mitochondria, and a combination thereof;

(ii) V12: the average number of individual mitochondria per unit of cell area, or a dispersion descriptor of the numbers of individual mitochondria per unit of cell area;

(iii) V13: the average area of mitochondria, or a dispersion descriptor of the areas of mitochondria;

(iv) V15: the frequency of mitochondria displaying an area between 0.51 and 1 μm²;

(v) V23: the frequency of mitochondria displaying an area between 10 and 20 μm²;

(vi) a variable selected from the group consisting of V29: the frequency of mitochondria displaying an area between 70 and 100 μm², V7: the total area of regions containing entwined mitochondria to the total cell area, and V30: the frequency of mitochondria displaying an area between 100 and 200 μm², and any combination thereof; and

(vii) a variable selected from the group consisting of V32: the average moving speed of mitochondria, or a dispersion descriptor of the moving speeds of mitochondria, and V33: the average maximal moving speed of individual mitochondria, or a dispersion descriptor of maximal moving speeds of individual mitochondria, and a combination thereof.

In an embodiment, the method further comprises providing a sample from a subject affected with HD.

The subject affected with HD may be at any stage of the disease, in particular in the asymptomatic stage or at early stage of the disease progression. Preferably, the subject comprises a mutation of the htt gene with at least 40 CAG repeats.

The screening method may be conducted on the sample of a HD patient in order to select a suitable therapy, i.e. personalized medicine, or on a population of HD samples, e.g. for drug development or drug repositioning.

Preferably, the sample is selected from the group consisting of skin biopsy, nervous tissue biopsy, serum and blood.

According to the nature of the sample, living cells may be selected from the group consisting of fibroblasts, lymphocytes and neuronal cells directly obtained from the sample or from primary cultures of cells from said sample. Living cells may also be induced pluripotent stem cells derived from adult somatic cells, in particular from fibroblasts obtained from the sample. Preferably, cells are non-transformed living cells to obtain results as close as possible of the in vivo situation.

In a preferred embodiment, the sample is a skin biopsy and cells are fibroblasts. In particular, the skin biopsy may be treated in order to isolate or enriched the culture in fibroblasts.

In an embodiment, the method further comprises, before measuring the values of mitochondrial behaviour variables, labelling mitochondria contained in living cells obtained from the sample. Preferably mitochondria are labelled before step a).

Mitochondria may be labeled using any method commonly known by the skilled person. Preferably, the label is a fluorescent, luminescent or colored label. More preferably, the label is a fluorescent label. Mitochondria may be labelled using a probe specific of said organelle and/or by transfection of a reporter gene (e.g. a GFP-expressing construct with mitochondrion-targeted expression) and/or by microinjection inside live cells of a marker or dye specifically taken up by said organelle. All these techniques are well known by the man skilled in the art and some commercial kits are available for this type of labelling and should be used according to manufacturer's recommendations. In particular, mitochondria may be labelled using calcein and cobalt (Petronilli et al., 1998), fluorescent rhodamine derivatives such as Rhodamine 123, tetramethylrhodamine methyl ester (TMRM) and tetramethylrhodamine ethyl ester (TMRE), carbocyanine dyes, 10-N-Nonyl acridine orange (NAO) or a MitoTracker dye, in particular MitoTracker Green (MTG) or MitoTracker red (CMXRos). Preferably, mitochondria are labelled using a dye which is not sensitive to mitochondrial membrane potential. In particular, the dye can be selected from the group consisting of MitoTracker Green (MTG), carbocyanine dyes, 10-N-Nonyl acridine orange (NAO) and the combination calcein-cobalt. In a preferred embodiment, mitochondria are labelled using MitoTracker Green.

In another embodiment, mitochondria are not labelled and the values of the mitochondrial behaviour variables are measured using a label-free microscopic technique such as differential interference contrast (DIC) microscopy.

In step a), living cells are contacted with a test compound.

The test compound may be selected from the group consisting of chemical compounds, biological compounds, radiations, and any combination thereof.

In an embodiment, the test compound is a radiation, in particular a radiation selected from the group consisting of X-rays, gamma rays, alpha particles, beta particles, photons, electrons, neutrons, radioisotopes, and other forms of ionizing radiation, and any combination thereof.

In another embodiment, the test compound is a chemical compound, i.e. an organic or inorganic compound. For example, the test compound may be a drug authorized to be marketed for another application than HD, a compound from a high-throughput chemical library or a nucleic acid construct suitable for gene therapy. If the test compound is a drug authorized to be marketed for another application than HD, the method may be used for drug repositioning.

In a further embodiment, the test compound is a biological compound. The biological compound may be selected from the group consisting of proteins, lipids, nucleic acids, carbohydrates and any other biological molecules or complexes. It may also be a therapeutic cell used for cell therapy or engineered virus for virotherapy. In particular, therapeutic cells may be stem cells, progenitor cells, mature and functional cells for cell replacement therapy or genetically modified cells for cell-based gene therapy.

The technique for contacting living cells with the test compound may vary according to the nature of said compound and may be easily chosen by the skilled person. In particular, if the test compound is a chemical or biological compound, it may be added to the cell culture medium. For cell therapy, living cells obtained from the sample and therapeutic or genetically modified cells may be contacted using a co-culture system allowing or not direct contact between the cells. If the test compound is radiation, cells in culture medium may be submitted to radiation. If the test compound is a nucleic acid construct, it may be added to the culture medium in a suitable vehicle such as liposome, transfected or directly injected into the cells. All these techniques are well known by the skilled person.

In step b) of the method, the values of several mitochondrial behavior variables are measured in cells contacted with the test compound in step a). As shown in the experimental section, these variables have been selected by the inventors to constitute a HD-signature that is sufficient to segregate cells from HD patients to cells from healthy patients and that can be reversed when cells are contacted with a test compound useful in the treatment or prevention of HD.

The values of mitochondrial behavior variables are measured by analyzing images of mitochondria, preferably labeled mitochondria, observed in living cells. Images were taken, for example, at least every 10 s at high scan speed for at least 2 min, preferably every 0.1 to 10 s at high scan speed for at least 2 to 6 min. The image capture may be carried out using any suitable microscopic technique such as fluorescent microscope or differential interference-contrast (DIC) microscope, coupled to an image acquisition system. In particular, if mitochondria are labeled, the image capture may be carried out using a fluorescent microscope. If mitochondria are not labeled, the image capture may be carried out using a DIC microscope. The image acquisition device may be any device allowing capture of high-resolution frames at high speed such as a

Charge-Coupled Device (CCD). In a preferred embodiment, the image capture is performed in three spatial dimensions. The image capture may be thus carried out using a microscope equipped with a motorized plate allowing the visualization of a sample in three dimensions. The measurements may be conducted in presence or after exposition to the test compound, preferably in presence of the test compound. Preferably, cells are kept at 37° C. during the image capture.

Mitochondria are mobile within the cell cytoplasm as they move along the cell microtubule and actin filament network and the first variable is selected from the group consisting of V1 and V34, and a combination thereof, wherein V1 is the average frequency of stops during trajectories of individual mitochondria and V34 is the average frequency of burst during displacement of individual mitochondria.

The variable V1 is the average frequency of stops during trajectories of individual mitochondria. This frequency is assessed by tracking each mitochondrion from frame to frame and recording the number of stops, i.e. the number of periods during which the mitochondrion remains immobile between two frames. The variable V1 is thus obtained by measuring the number of stops per unit time for each traced mitochondrion and calculating the average frequency of stops from data of all traced mitochondria.

The variable V34 is the average frequency of burst during displacement of individual mitochondria. This frequency is assessed by tracking each mitochondrion from frame to frame and recording the number of burst, i.e. the number of sudden displacements within 30% of the maximal displacements of the mitochondrion during the period of capture. The variable V34 is thus obtained by measuring the number of burst per unit time for each traced mitochondrion and calculating the average frequency of burst from data of all traced mitochondria.

The second variable, V12, is the average number of individual mitochondria per unit of cell area, or a dispersion descriptor of the numbers of individual mitochondria per unit of cell area. This number is preferably determined using an image analysis software by counting each mitochondrion on an image of the cell and expressing the result in number per unit of cell area, preferably per μm² of cell area. The variable may be obtained by calculating the average number, of mitochondria per unit of cell from several measurements. This variable may also be obtained by calculating a dispersion descriptor, preferably selected from the group consisting of the variance, the standard deviation and an interquantile range, more preferably selected from the group consisting of the variance, the standard deviation and the interquartile range.

The third variable, V13, is the average area of mitochondria, or a dispersion descriptor of the areas of mitochondria. The variable may be obtained by determining the area of each mitochondrion and calculating the average area from data of all observed mitochondria. The variable may also be obtained by determining the area of each mitochondrion and calculating a dispersion descriptor of the areas of mitochondria. Preferably the descriptor selected from the group consisting of the variance, the standard deviation and an interquantile range, more preferably selected from the group consisting of the variance, the standard deviation and the interquartile range. Preferably, the area of each mitochondrion is determined using an image analysis software.

The fourth variable, V15, is the frequency of mitochondria displaying an area between 0.51 and 1 μm².

The 5^{th} variable, V23, is the frequency of mitochondria displaying an area between 10 and 20 μm².

The 6^{th} variable is selected from the group consisting of V29, V7 and V30, and any combination thereof, wherein V29 is the frequency of mitochondria displaying an area between 70 and 100 μm², V7 is the total area of regions containing entwined mitochondria to the total cell area, and V30 is the frequency of mitochondria displaying an area between 100 and 200 μm².

The variables V15, V23, V29 and V30 relate to the frequency of mitochondria displaying a specific range of area. Preferably, the area of each mitochondrion is determined using image analysis software. The variables V15, V23, V29 and V30 are thus obtained by measuring the area of mitochondria and determining the number of mitochondria displaying an area between 0.51 and 1 μm², 10 and 20 μm², 70 and 100 μm² and 100 and 200 μm², respectively. The results are then expressed in percent of the total number of mitochondria.

The variable V7 is the total area of regions containing tangled mitochondria, i.e. mitochondria that are interlaced to a point where individualisation of single mitochondria is impossible, to the total cell area. This variable assesses the concordance between the directionality of mitochondria with that of the cytoskeleton and is correlated to the state of the relationship between mitochondria and the cytoskeleton.

The 7^{th} variable is selected from the group consisting of V32 and V33, and a combination thereof, wherein V32 is the average moving speed of mitochondria, or a dispersion descriptor of moving speeds of mitochondria, and V33 is the average maximal moving speed of individual mitochondria, or a dispersion descriptor of maximal moving speeds of mitochondria.

The variable V32 is the average moving speed of mitochondria. The moving speed of mitochondria is assessed by tracking each mitochondrion from frame to frame and recording the average speed of each mitochondrion. The variable V32 may be then obtained by calculating the average moving speed of mitochondria from data of all traced mitochondria. The variable may also be obtained by calculating a dispersion descriptor of the moving speeds of mitochondria. Preferably the descriptor selected from the group consisting of the variance, the standard deviation and an interquantile range, more preferably selected from the group consisting of the variance, the standard deviation and the interquartile range.

The variable V33 is the average maximal moving speed of individual mitochondria. The maximal speed is assessed by tracking each mitochondrion from frame to frame and recording the maximal speed of the mitochondrion reached during the capture. The variable V33 may be then obtained by calculating the average maximal moving speed of mitochondria from data of all traced mitochondria. The variable may also be obtained by calculating a dispersion descriptor of the maximal moving speeds of mitochondria. Preferably the descriptor selected from the group consisting of the variance, the standard deviation and an interquantile range, more preferably selected from the group consisting of the variance, the standard deviation and the interquartile range.

In a preferred embodiment, a recording and data management device, e.g. a computer with a suitable software, is used to record and analyze images of mitochondria observed through the microscope.

The number of mitochondria and cells to be analyzed for each variable is easily determined by the skilled person using statistic methods. Preferably, at least 50, 80 or 100 mitochondria are analyzed for each variable, preferably from at least 3, 10 or 15 cells. In a particular embodiment, at least 50, 80 or 100 mitochondria are analyzed for variables V1 and V32 and at least 100, 250, 500, 800, 900 or 1000 mitochondria are analyzed for variables V12, V13, V15, V23 and V29.

The method may comprise measuring the values of a combination of variables selected from the group consisting of V1, V12, V13, V15, V23, V29 and V32; V1, V12, V13, V15, V23, V29 and V33; V1, V12, V13, V15, V23, V7 and V32; V1, V12, V13, V15, V23, V7 and V33; V1, V12, V13, V15, V23, V30 and V32; V1, V12, V13, V15, V23, V30 and V33; V34, V12, V13, V15, V23, V29 and V32; V34, V12, V13, V15, V23, V29 and V33; V34, V12, V13, V15, V23, V7 and V32; V34, V12, V13, V15, V23, V7 and V33; V34, V12, V13, V15, V23, V30 and V32; and V34, V12, V13, V15, V23, V30 and V33, wherein V1, V34, V12, V13, V15, V23, V29, V7, V30, V32 and V33 are as defined above.

In a particular embodiment, the method comprises measuring the variables V1, V12, V13, V15, V23, V29 and V32 as defined above. In this embodiment, the method may further comprise measuring at least one additional variable selected from the group consisting of V7, V30 V33 and V34, as defined above.

Alternatively, the variables can be measured on samples from a population of HD patients. The values obtained for each variable are then averaged.

Each variable may be weighted in order to adjust their importance.

The method of screening of the invention may further comprise comparing the values of the variables obtained in step b) in presence of the test compound with the values obtained in absence of the test compound. Preferably, the values in absence of the test compound are obtained on cells from the same sample than in step b) before contacting the test compound. In a particular embodiment, these values are obtained after labelling mitochondria and before step a), i.e. on cells with labelled mitochondria before contacting the test compound. The value of each variable is measured as detailed above. Values obtained without the test compound may be used as negative control to identify compounds that could be useful in the treatment of HD.

The method may also further comprise comparing the values of variables obtained in presence, and optionally in absence of the test compound, with the values of said variables measured in a sample obtained from a healthy subject (in absence of the test compound). Preferably, the healthy subject is about the same age as the HD patient providing the HD sample. The value of each variable is measured as detailed above. Values obtained from the sample from the healthy subject may be used as positive control to identify compounds that could be useful in the treatment of HD. Alternatively, this positive control can be obtained by measuring the variables on samples from a population of healthy subject. The values obtained for each variable are then averaged.

In a particular embodiment, the values of variables of the HD signature are measured in presence of several concentrations of the test compound in order to determine the dose-response effect of the test compound on HD.

The significance of differences of measured values may be determined using any suitable statistic test such as ANOVA.

Using a discriminating equation, the values obtained for the variables for each dose of the test compound, may be represented as a score. The effect of each dose may be evaluated in respect to the score obtained for the negative and/or positive controls.

In particular, the score for each variable may be calculated using the following ratio:

Score of the variable Vx=(NC−Var)*100/(NC−PC)

(NC: value or average value of the negative control; PC: value or average value of the positive control; Var: value of the variable).

A global percentage of phenotypic rescue may be obtained by adding up the scores of each measured variable.

The results may thus be expressed as a percentage of phenotypic rescue, the positive control (healthy sample) being 100% and the negative control (HD sample in absence of the test compound) being 0%. Test compound providing a positive phenotypic rescue, i.e. a compound that is able to partially or totally reverse the HD signature, is identified as potentially useful in the treatment of HD. In a particular embodiment, a test compound is identified as potentially useful if the phenotypic rescue is above 50%, more preferably above 60%, 70%, 80% or 90%.

Preferably, a test compound is identified as potentially useful in the treatment of HD if all measured variables have a positive score, i.e. if a rescue is observed for each variable.

In another aspect, the present invention concerns a method for diagnosing Huntington disease in a subject, wherein the method comprises measuring in living cells obtained from a sample from said subject, the values of the mitochondrial behaviour variables (i) to (vii):

(i) a variable selected from the group consisting of V1: the average frequency of stops during trajectories of individual mitochondria and V34: the average frequency of burst during displacement of individual mitochondria, and a combination thereof;

(ii) V12: the average number of individual mitochondria per unit of cell area, or a dispersion descriptor of the numbers of individual mitochondria per unit of cell area;

(iii) V13: the average area of mitochondria, or a dispersion descriptor of the areas of mitochondria;

(iv) V15: the frequency of mitochondria displaying an area between 0.51 and 1 μm²;

(v) V23: the frequency of mitochondria displaying an area between 10 and 20 μm²;

(vi) a variable selected from the group consisting of V29: the frequency of mitochondria displaying an area between 70 and 100 μm², V7: the total area of regions containing entwined mitochondria to the total cell area, and V30: the frequency of mitochondria displaying an area between 100 and 200 μm², and any combination thereof; and

(vii) a variable selected from the group consisting of V32: the average moving speed of mitochondria, or a dispersion descriptor of the moving speeds of mitochondria, and V33: the average maximal moving speed of individual mitochondria, or a dispersion descriptor of the maximal moving speeds of mitochondria, and a combination thereof.

All embodiments described above for the method of screening are also contemplated in this aspect.

The method may further comprise providing a sample from the subject.

Preferably, mitochondria contained in living cells are labelled before measuring mitochondrial behavior variables.

The subject may have clinical signs that resemble HD or may be without any symptom.

The method may further comprise conducting a genetic test to determine the number of CAG repeats in the htt gene.

In a preferred embodiment, the subject comprises 36 to 39 CAG repeats in the htt gene. In this case, the method of the invention is particularly relevant due to the impossibility to predict the penetrance of the disease from the number of CAG repeats.

The method may further comprise determining if said subject is affected with Huntington disease based on the measured values of mitochondrial behaviour variables. The diagnosis of HD may be obtained by comparing the score obtained with the sample of the subject with the score of the sample obtained from a healthy patient or from a HD patient, or alternatively obtained from a population of healthy subjects or HD patients.

A gain of function in variables V12, V15 and the 1^{st} variable, i.e. V1 and/or V34, and a loss of function in variables V13, V23, the 6^{th} variable, i.e. V29, V7 and/or V30, and the 7^{th} variable, i.e. V32 and/or V33, by comparison with the values of these variables obtained from a healthy sample, is indicative of HD.

In a particular embodiment, the method comprises measuring the mitochondrial behaviour variables V1, V12, V13, V15, V23, V29 and V32 as defined above, a gain of function in variables V1, V12 and V15 and a loss of function in variables V13, V23, V29 and V32, by comparison with the values of these variables obtained from a healthy sample, is indicative of HD. The method may further comprise calculating the z-scores of measured variables. In particular, in HD sample the z-scores of variables V1, V12 and V15 are positive and the z-scores of variables V13, V23, V29 and V32 are negative.

In another particular embodiment, the method comprises measuring the mitochondrial behaviour variables V1, V23 and V32 as defined above, a gain of function in variables V1 and V23 and a loss of function in variable V32, by comparison with the values of these variables obtained from a healthy sample, is indicative of HD. In this embodiment, the method may further comprise calculating the z-scores of measured variables. In particular, in HD sample the z-scores of variables V1, V23 and V32 are positive and the z-scores of variable V32 is negative. The method may further comprise measuring the mitochondrial behaviour variables V13, V12, V15 and/or V29.

The present invention also concerns a method for providing useful information for the diagnosis of Huntington disease in a subject, wherein the method comprises measuring in living cells obtained from a sample from said subject the values of the mitochondrial behaviour variables (i) to (vii):

(i) a variable selected from the group consisting of V1: the average frequency of stops during trajectories of individual mitochondria and V34: the average frequency of burst during displacement of individual mitochondria, and a combination thereof;

(ii) V12: the average number of individual mitochondria per unit of cell area, or a dispersion descriptor of the numbers of individual mitochondria per unit of cell area;

(iii) V13: the average area of mitochondria, or a dispersion descriptor of the areas of mitochondria;

(iv) V15: the frequency of mitochondria displaying an area between 0.51 and 1 μm²;

(v) V23: the frequency of mitochondria displaying an area between 10 and 20 μm²;

(vi) a variable selected from the group consisting of V29: the frequency of mitochondria displaying an area between 70 and 100 μm², V7: the total area of regions containing entwined mitochondria to the total cell area, and V30: the frequency of mitochondria displaying an area between 100 and 200 μm², and any combination thereof; and

(vii) a variable selected from the group consisting of V32: the average moving speed of mitochondria, or a dispersion descriptor of the moving speeds of mitochondria, and V33: the average maximal moving speed of individual mitochondria, or a dispersion descriptor of the maximal moving speeds of mitochondria, and a combination thereof.

All embodiments described above are also contemplated in this aspect.

In another aspect, the present invention also concerns a method for monitoring the response of a subject affected with Huntington disease to therapy, or for selecting a subject affected with Huntington disease for therapy, wherein the method comprises

a) measuring in living cells obtained from a sample from said subject, before and after the administration of the treatment, the values of the mitochondrial behaviour variables (i) to (vii):

(i) a variable selected from the group consisting of V1: the average frequency of stops during trajectories of individual mitochondria and V34: the average frequency of burst during displacement of individual mitochondria, and a combination thereof;

(ii) V12: the average number of individual mitochondria per unit of cell area, or a dispersion descriptor of the numbers of individual mitochondria per unit of cell area;

(iii) V13: the average area of mitochondria, or a dispersion descriptor of the areas of mitochondria;

(iv) V15: the frequency of mitochondria displaying an area between 0.51 and 1 μm²;

(v) V23: the frequency of mitochondria displaying an area between 10 and 20 μm²;

(vi) a variable selected from the group consisting of V29: the frequency of mitochondria displaying an area between 70 and 100 μm², V7: the total area of regions containing entwined mitochondria to the total cell area, and V30: the frequency of mitochondria displaying an area between 100 and 200 μm², and any combination thereof; and

vii) a variable selected from the group consisting of V32: the average moving speed of mitochondria, or a dispersion descriptor of the moving speeds of mitochondria, and V33: the average maximal moving speed of individual mitochondria, or a dispersion descriptor of the maximal moving speeds of mitochondria, and a combination thereof; and

b) comparing the measured values obtained before and after the administration of the treatment in step a).

All embodiments described above for the method of screening are also contemplated in this aspect.

Preferably, mitochondria contained in living cells are labelled before measuring mitochondrial behavior variables.

The method may further comprise providing a sample from the subject before and/or after the administration of the treatment, preferably before and after the treatment.

The therapy may comprise administering one or several chemical or biological compounds, as well as radiations, as defined above.

As explained above for the method of screening, the values obtained for the mitochondrial behaviour variables before and after the administration of the treatment may be represented as a score. The effect of the treatment may be thus evaluated by comparing the scores obtained before and after the treatment. Optionally, the scores may also be compared with the score obtained from a healthy sample or from a population of healthy samples.

In a preferred embodiment, a score is calculated for each variable using the following equation: score=(NC−Var)*100/(NC−PC), wherein NC is the value obtained before the administration of the treatment, PC is the value or average value obtained with healthy sample(s), and Var is the measured value of the variable. The patient is responsive to the therapy or is susceptible to benefit from the therapy when all measured variables have a positive score. If one or several variables have negative scores, the therapy may worsen the symptoms of the disease and should be stopped or avoided.

The results may also be expressed as a percentage of phenotypic rescue, the positive control (healthy sample) being 100% and the negative control (HD sample without any treatment, e.g. the HD sample obtained before the treatment) being 0%. A positive phenotypic rescue is indicative that the HD patient is responsive to the therapy or is susceptible to benefit from the therapy. On the contrary, a negative phenotypic rescue indicates that the therapy may worsen the symptoms of the disease and should be stopped or avoided.

Further aspects and advantages of the present invention will be described in the following examples, which should be regarded as illustrative and not limiting.

### EXAMPLES

### Example 1

### Huntington Disease Signature

Skin Biopsy Sample Collection

Skin biopsies were collected from 12 human donors comprising six healthy subjects and six HD patients (Table 1). No other disease or co-morbidity was reported in the two groups.

The cohort was chosen to minimize possible confounding age and gender effects by selecting only female subjects and by limiting the age range within roughly a decade around 36 or 38 in healthy and diseased subjects, respectively.

This selection also minimized the heterogeneity in the healthy population.

Among HD patients, the duration of clinical manifestations of the disease prior to biopsy differed from one to ten years. The clinical manifestation profiles were available for 4 HD patients out of 6. Chorea was present in two patients at different level of severity. One patient had a hypokinesic variant of HD with dystonia and marked tremor. One was marginally dystonic and showed bradykinesia and hypomimia, and another had bradykinesia. The size of the CAG repeat was available for 4 HD patients and ranged from 47 to 69 CAG repeats on the mutant allele. One patient is homozygote and severely affected.

TABLE 1
Cohort characteristics

| ID of the | | Age at the date of | Onset of the |
|---|---|---|---|
| sample | Status | biopsy | disease |
| 071203 | Apparently healthy | 20 | |
| 080401 | Apparently healthy | 47 | |
| 090801 | Apparently healthy | 45 | |
| 071201 | Apparently healthy | 31 | |
| 071204 | Apparently healthy | 21 | |
| 090407 | Apparently healthy | 54 | |
| 090402 | HD - severe | 37 | 27 |
| 090404 | HD - mild | 44 (asymptomatic) | 60 |
| 090403 | HD - average severity | 43 | 42 |
| 090405 | HD - average severity | 56 | 46 |
| 090406 | HD - severe | 20 | 14 |
| 090401 | HD - severe | 29 | 18 |

Cell Culture

Patient-derived fibroblasts were cultured in DMEM containing 15% fetal calf serum. Cells of the sample were expanded and found stable for at least 16-20 passages.

Dynamical Imaging

Fibroblasts were labelled with MitoTracker green, a cell-permeant mitochondrial dye not sensitive to mitochondrial membrane potential, for 30 minutes. Images were recorded from an epifluorescence microscope continuously for 6 minutes. Cells were kept at 37° C. for the duration of the image capture. For each experimental condition, 3 to 15 cells were recorded per well from three independent plates, i.e. up to 6000 individual mitochondria. The maximum duration for data acquisition was 30 minutes (i.e. five cells observed during 6 min). Images were captured with a Zeiss Axioplan II microscope along three dimensions in space and in time.

Identification of Markers of the HD Signature

37 variables defining mitochondrial behaviour and labelled V1 to V37 were simultaneously measured. These variables reflected the mitochondrial motility, the mitochondrial morphology, the mitochondrial reticular network or relationship with the cytoskeleton and the mitochondrial permeability.

Using statistical methods (Principal Component Analysis, Hierarchical cluster classification, Discriminant Analysis and ANOVA), seven variables (V1, V32, V12, V13, V15, V23 and V29) were found to be sufficient to segregate healthy and HD samples with only one misclassification (ID 090407).

V1: average frequency of stops during trajectories of individual mitochondria;

V12: average number of individual mitochondria per unit of cell area, or variance, standard deviation or interquartile range of the measured values;

V13: average area of mitochondria or variance, standard deviation or interquartile range of the measured values;

V15: frequency of mitochondria displaying an area between 0.51 and 1 μm²;

V23: frequency of mitochondria displaying an area between 10 and 20 μm²;

V29: frequency of mitochondria displaying an area between 70 and 100 μm²;

V32: average moving speed of mitochondria, or variance, standard deviation or interquartile range of the measured values;

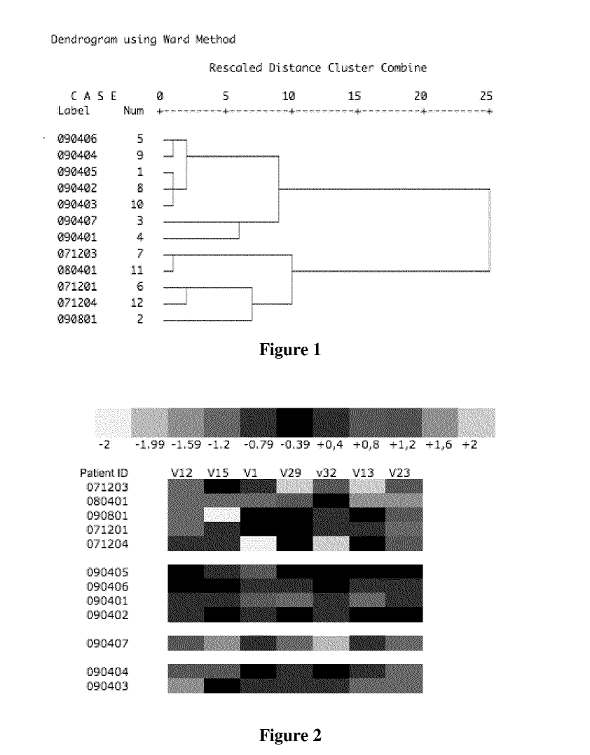

The dendogram of the hierarchical cluster analysis applying the Ward's method and obtained with the seven variables is shown in FIG. 1. The cluster analysis shows two subgroups composed of the diseased patient (top group) in which subject 090407 is misplaced, and a group of healthy subjects (bottom group).

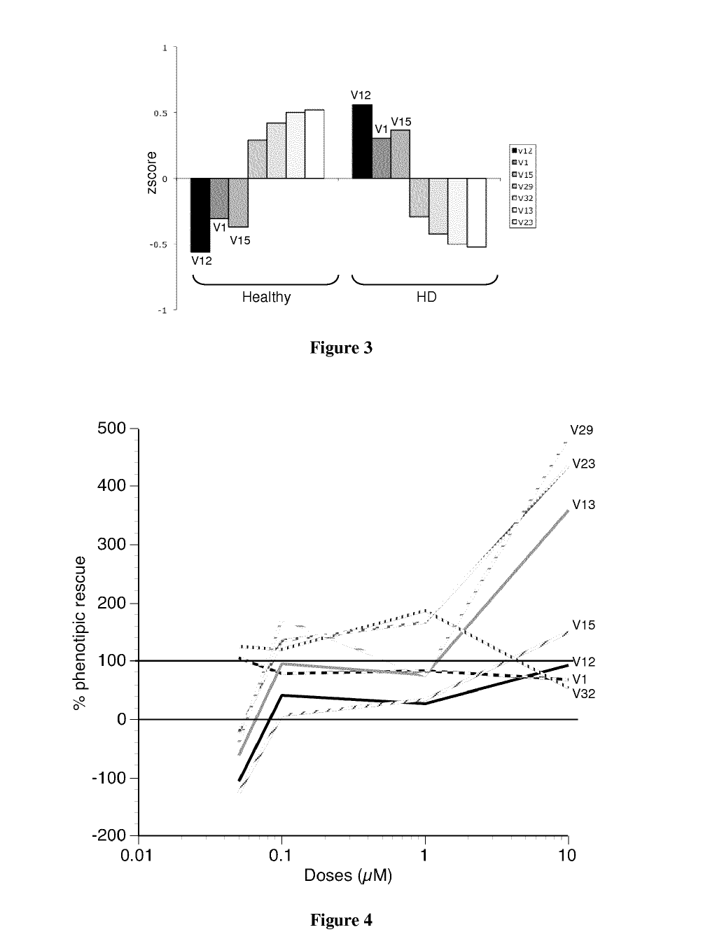

For each patient of the cohort, the number of mitochondria studied for each variable was from 59 to 100 for V1 and V32, and from 970 to 3696 for V12, V13, V15, V23 and V29. From these results, a heat map corresponding to the actual values obtained in the subjects was derived (FIG. 2). This map shows a gain of function for variables V1, V12 and V15 and a loss of function for variables V13, V23, V29 and V32 for HD patients and the inverse pattern for healthy subjects (except for the subject 090407) (FIG. 3).

The robustness of the signature comprising the seven variables was confirmed by analyzing about 39,000 additional mitochondria from 215 cells in 9 independent experiments and repeated the hierarchical cluster analysis.

### Example 2

### Reversal of the HD-Signature

Resveratrol is a plant polyphenol found in grapes and red wine. Resveratrol is associated with beneficial effects on aging, metabolic disorders, inflammation and cancer. Despite poor bioavailability, resveratrol was shown to rescue mutant huntingtin polyglutamine toxicity in several in vitro and in vivo models mimicking HD (Parker et al., 2005; Maher et al., 2011; Ho et al., 2010). Resveratrol may exert its effects by targeting several key metabolic sensor/effector proteins, such as AMPK, SIRT1, and PGC-1α (Pasinetti et al., 2011).

Cyclosporine A (CSA) is an anti-inflammatory drug inhibiting calcineurin. Calcineurin is a Ca²⁺- and calmodulin-dependent protein serine-threonine phosphatase that is thought to play an important role in the neuronal response to changes in the intracellular Ca²⁺ concentration. Altered mitochondrial membrane potential and aberrant Ca²⁺ handling are molecular mechanisms associated with HD and are targets of CSA (Choo et al., 2004). CSA and FK506, another calcineurin inhibitor, have been shown to have controversial effects on different animal models mimicking HD (Pineda et al., 2009; Kumar et al., 2010; Hernandez-Espinosa et al., 2006).

Materials and Methods

Patient-derived fibroblasts were cultured in DMEM containing 15% fetal calf serum. and 0.5% DMSO, a concentration known to be inert on the mitochondrial behaviour.

Cells were incubated with different doses of resveratrol or cyclosporine A (CSA) diluted in DMSO or with the vehicle only (DMSO) for 30 minutes and observed through time-lapse video-microscopy for another 30 minutes during dynamic image capture.

Resveratrol and CSA were tested at 4 doses spanning 3.5 log (10, 1, 0.1 and 0.05 μM).

The negative control was cells from HD patient (ID 090401) with the vehicle only and the positive control was cells from healthy patient (ID 071201) with the vehicle only.

The raw data obtained for each mitochondrion were expressed as a ratio (%) according to the following formula: DATA=(NC−Var)*100/(NC−PC) (NC=average value of the negative control; PC=average value of the positive control; Var=value of the variable).

Results

Resveratrol

Dose-response analysis of the effect of Resveratrol on the 7 variables constituting the HD-signature is shown in FIG. 4. Effects are expressed as a percent of control values as defined above, i.e. % of phenotypic rescue. 0% represents the diseased status while 100% represents the healthy status.

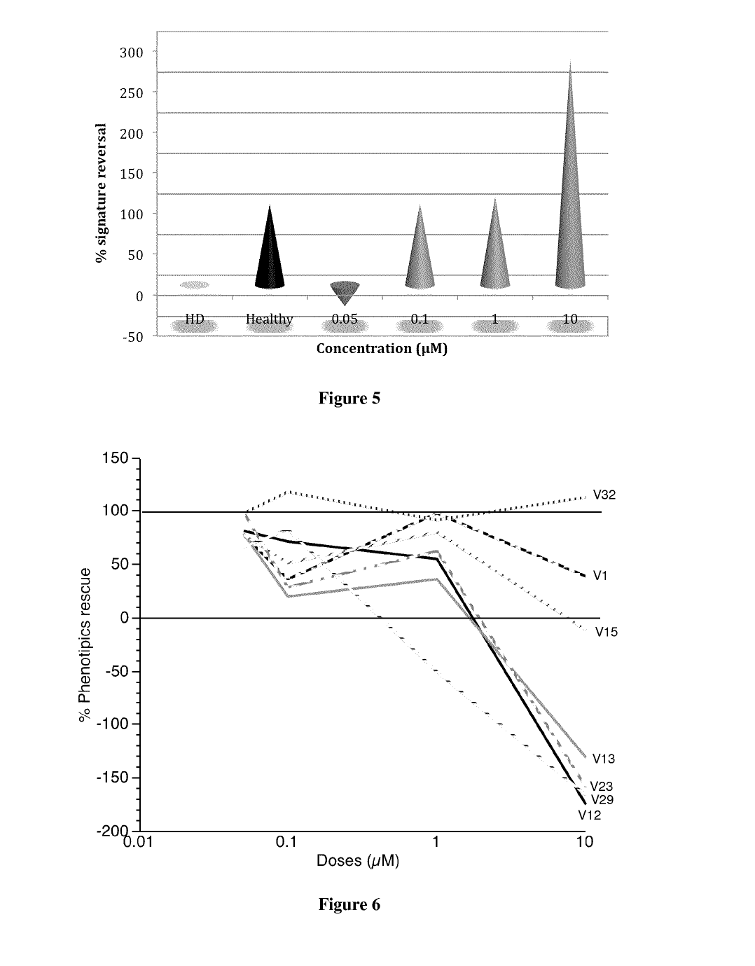

To express the weighted effect of the variables on the overall rescue, a discriminant equation was applied. The resulting activity profile (FIG. 5) shows that 0.05 μM resveratrol provides a detrimental effect with a mild worsening of the diseased status while 0.1 and 1 μM provide 86 and 89.9% recovery, respectively. At 10 μM, the effect exceeds the healthy status. and may indicate over compensation.

The overall effect of Resveratrol thus shows disease-modifying capabilities and rescue of the disease status except at low dose.

Cyclosporin A (CSA)

Dose-response analysis of the effect of CSA on the 7 variables constituting the HD-signature is shown in FIG. 6. Effects are expressed as a percent of control values as defined above, i.e. % of phenotypic rescue. 0% represents the diseased status while 100% represents the healthy status.

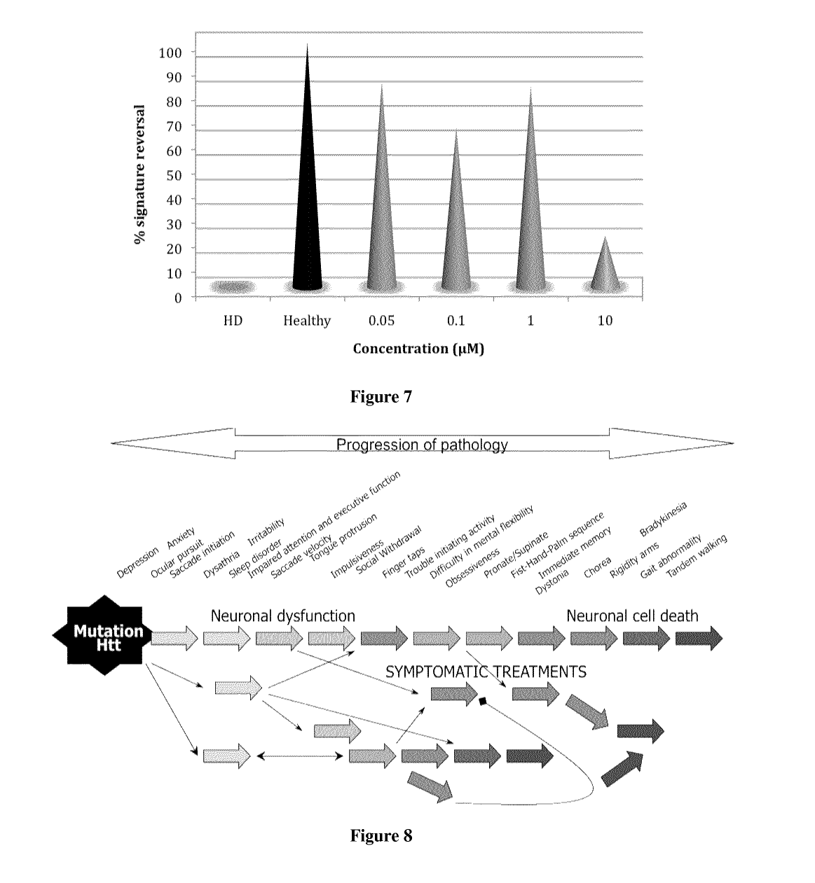

To express the weighted effect of the variables on the overall rescue, a discriminant equation was applied. The resulting activity profile (FIG. 7) shows dose-dependent loss of efficacy in the overall rescue of the diseased phenotype treated with CSA. Worsening of the beneficial effects of CSA at high dose (10 μM) may reflect its toxicity potential.

However, at 0.05 μM, CSA provides nearly 84% recovery, which makes it a more potent drug than Resveratrol.

### CONCLUSION

These results show that the HD signature established by the inventors and comprising the 7 variables is sufficient to segregate HD patients and healthy patients and may be used as tool for the diagnosis of HD at different stages of the disease progression including pre-symptomatic stages.

Using Resveratrol and Cyclosporine A, two compounds previously shown to have disease reversal capability in animal models, the inventors have also demonstrated that this HD signature can be reversed and thus allows the study of disease-modifying properties of compounds even in a dose-dependent manner. This signature can thus be used as a powerful tool to screen and identify novel drugs useful for HD treatment.

Because one can monitor the evolution of this signature prior or after administration of a compound in a HD patient, this signature can also be used as a surrogate marker for treatment efficacy, in particular in clinical trials.

### REFERENCES

Carter R J, et al. J Neurosci. 1999 Apr. 15; 19(8):3248-57.
Choo Y S, et al. Hum Mol Genet. 2004 Jul. 15; 13(14):1407-20.
Faber P W, et al., 2002, PNAS USA, 99(26):17131-17136.
Gray M, et al. J Neurosci. 2008 Jun. 11; 28(24):6182-95.
Hernández-Espinosa D and Morton A J. 2006, Brain Res Bull.; 69(6):669-79.
Ho D J, et al. Exp Neurol. 2010 September; 225(1):74-84.
Hodgson J G, et al. Neuron. 1999; 23:181-192.
Jackson G R, et al., Neuron, 1998, 21(3): 633-642.
Jacobsen J C, et al., Human Molecular Genetics, 2010, 19(10): 1873-1882,
Kumar P, et al. 2010, Int J Toxicol. 29(3):318-25.
Maher P, et al., Hum Mol Genet. 2011 Jan. 15; 20(2):261-70.
Martino D. et al. J Neurol Neurosurg Psychiatry. 2012 Sep. 19
Menalled L B, et al. J Neurosci. 2002; 22:8266-8276.
Parker, J. A., et al., 2005, Nat Genet. 37: 349-350.
Pasinetti G. M., et al., Exp Neurol. 2011 November; 232(1):1-6.
Petronilli V, et al., 1998, Biofactors. 8(3-4):263-72;
Pineda J R, et al. 2009 Mol Brain. 27; 2:33.
Politis, et al. 2011, Hum. Brain Mapp. 32, 258-270.
Tabrizi S J, et al. Ann Neurol. 2000 January; 47(1):80-6.
Trushina E, et al. Mol Cell Biol. 2004; 24:8195-8209.
Wheeler V C, et al. Hum Mol Genet. 2000; 9:503-513.
Yang D, et al., Human Molecular Genetics, 2010 19(20): 3983-3994.
Yang S H, al., Nature, 2008, 453(7197):921-924.

1-21. (canceled)

22. An in vitro method of screening for compounds useful in the treatment of Huntington disease, wherein the method comprises
a) contacting living cells obtained from a sample from a subject affected with Huntington disease with a test compound; and
b) measuring in said contacted cells, the values of the mitochondrial behaviour variables (i) to (vii):
(i) a variable selected from the group consisting of the average frequency of stops during trajectories of individual mitochondria (V1), the average frequency of burst during displacement of individual mitochondria (V34), and a combination thereof;
(ii) the average number of individual mitochondria per unit of cell area, or a dispersion descriptor of the numbers of individual mitochondria per unit of cell area (V12);
(iii) the average area of mitochondria, or a dispersion descriptor of the areas of mitochondria (V13);
(iv) the frequency of mitochondria displaying an area between 0.51 and 1 μm² (V15);
(v) the frequency of mitochondria displaying an area between 10 and 20 μm² (V23);
(vi) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(vii) a variable selected from the group consisting of the average moving speed of mitochondria, or a dispersion descriptor of the moving speeds of mitochondria (V32) and the average maximal moving speed of individual mitochondria, or a dispersion descriptor of the maximal moving speeds of mitochondria (V33), and a combination thereof.

23. The method according to claim 22, further comprising comparing the values obtained in step b) with the values obtained in the absence of said test compound.

24. The method according to claim 22, further comprising calculating a score for each variable using the following equation:
score=(NC−Var)*100/(NC−PC),
wherein NC is the value or average value obtained with HD sample(s) in the absence of the test compound, PC is the value or average value obtained with healthy sample(s), and Var is the measured value of the variable.

25. The method according to claim 24, wherein a test compound is identified as useful in the treatment of Huntington disease when all measured variables have a positive score.

26. The method according to claim 22, wherein the method comprises measuring the mitochondrial behaviour variables V1, V12, V13, V15, V23, V29 and V32.

27. The method according to claim 26, wherein the method further comprises measuring at least one additional mitochondrial behaviour variable selected from the group consisting of V7, V30 V33 and V34.

28. The method according to claim 22, wherein the method further comprises comparing the measured values of the mitochondrial behaviour variables with the values of said variables measured in a sample obtained from a healthy subject.

29. The method according to claim 22, wherein the sample is selected from the group consisting of a skin biopsy sample, nervous tissue biopsy sample and serum or blood sample.

30. The method according to claim 22, wherein the living cells are selected from the group consisting of fibroblasts, induced pluripotent stem cells derived from fibroblasts, lymphocytes and neuronal cells.

31. The method according to claim 30, wherein the living cells are fibroblasts.

32. The method according to claim 22, wherein the dispersion descriptor is selected from the group consisting of the variance, the standard deviation and an interquantile range.

33. The method according to claim 22, wherein the mitochondria contained in living cells are labeled before measuring the values of the mitochondria behaviour variables.

34. The method according to claim 33, wherein the mitochondria in living cells are labeled with a fluorescent label.

35. The method according to claim 22, wherein the values of the mitochondrial behaviour variables are obtained from images captured using a fluorescence microscope or DIC microscope coupled to an image acquisition device.

36. An in vitro method for diagnosing Huntington disease in a subject, wherein the method comprises measuring in living cells obtained from a sample from said subject the values of the mitochondria behaviour variables (i) to (vii):
(i) a variable selected from the group consisting of the average frequency of stops during trajectories of individual mitochondria (V1), the average frequency of burst during displacement of individual mitochondria (V34), and a combination thereof;
(ii) the average number of individual mitochondria per unit of cell area, or a dispersion descriptor of the numbers of individual mitochondria per unit of cell area (V12);
(iii) the average area of mitochondria, or a dispersion descriptor of the areas of mitochondria (V13);
(iv) the frequency of mitochondria displaying an area between 0.51 and 1 μm² (V15);
(v) the frequency of mitochondria displaying an area between 10 and 20 μm² (V23);
(vi) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(vii) a variable selected from the group consisting of the average moving speed of mitochondria, a dispersion descriptor of the moving speeds of mitochondria (V32) and the average maximal moving speed of individual mitochondria, or a dispersion descriptor of the maximal moving speeds of mitochondria (V33), and a combination thereof.

37. The method according to claim 36, wherein the subject comprises 36 to 39 CAG repeats in the hit gene.

38. The method according to claim 36, further comprising calculating the z-scores of the measured variables.

39. The method according to claim 38, wherein positive z-scores of the 1^{st}, 2^{nd} and 4^{th} variables and negative z-scores of the 3^{rd}, 5^{th} and 7^{th} variables are indicative that the subject suffers from Huntington disease.

40. An in vitro method for monitoring the response of a subject affected with Huntington disease to therapy, wherein the method comprises:
a) measuring in living cells obtained from a sample from said subject, before and after the administration of the treatment, the values of the mitochondrial behaviour variables (i) to (vii):
(i) a variable selected from the group consisting of the average frequency of stops during trajectories of individual mitochondria (V1), the average frequency of burst during displacement of individual mitochondria (V34), and a combination thereof;
(ii) the average number of individual mitochondria per unit of cell area, or a dispersion descriptor of the numbers of individual mitochondria per unit of cell area (V12);
(iii) the average area of mitochondria, or a dispersion descriptor of the areas of mitochondria (V13);
(iv) the frequency of mitochondria displaying an area between 0.51 and 1 μm² (V15);
(v) the frequency of mitochondria displaying an area between 10 and 20 μm² (V23);
(vi) a variable selected from the group consisting of the frequency of mitochondria displaying an area between 70 and 100 μm² (V29), the total area of regions containing entwined mitochondria to the total cell area (V7), the frequency of mitochondria displaying an area between 100 and 200 μm² (V30), and any combination thereof; and
(vii) a variable selected from the group consisting of the average moving speed of mitochondria, a dispersion descriptor of the moving speeds of mitochondria (V32) and the average maximal moving speed of individual mitochondria, or a dispersion descriptor of the maximal moving speeds of mitochondria (V33), and a combination thereof; and
b) comparing the measured values obtained before and after the administration of the treatment in step a).

41. The method according to claim 40, further comprising calculating a score for each variable using the following equation:
score=(NC−Var)*100/(NC−PC),
wherein NC is the value obtained before the administration of the treatment, PC is the value or average value obtained with healthy sample(s), and Var is the measured value of the variable.

42. The method according to claim 10, wherein the patient is responsive to the therapy or is susceptible to benefit from the therapy when all measured variables have a positive score.
